SEQUENCE LISTING (6 sequences)

SEQ ID NO: 1 - DNA, 32 bp, Artificial sequence
  Synthetic nucleic acid sequence
ggtggcgggtgtggcatacgatcgacgtatgc

SEQ ID NO: 2 - DNA, 75 bp, Artificial sequence
  Synthetic nucleic acid sequence
gcatacgtcgatcgtatgaccggcaacgaaggaggagctgcgctgcaaagaagctgtcgctagacgatcgactac

SEQ ID NO: 3 - DNA, 30 bp, Artificial sequence
  Synthetic nucleic acid sequence
gtagtcgatcgtctagcggctcgcgggtgg

SEQ ID NO: 4 - DNA, 65 bp, Artificial sequence
  Synthetic nucleic acid sequence
ccacccgcgagcacagcttctttgcagcgcagctcctcgttcgttgccggtccacacccgccacc

SEQ ID NO: 5 - DNA, 32 bp, Artificial sequence
  Synthetic nucleic acid sequence
ggtggcgggtgtgggcagtattcgacgtatgc

SEQ ID NO: 6 - DNA, 30 bp, Artificial sequence
  Synthetic nucleic acid sequence
gtagtcgatcggcagtatgctcgcgggtgg

(19) United States
(12) Patent Application Publication
(10) Pub. No.: US 2014/0255924 A1
(43) Pub. Date: Sep. 11, 2014

(54) TWIST-TIE OLIGONUCLEOTIDE PROBES

(71) Applicant: AGILENT TECHNOLOGIES, INC., Loveland, CO (US)

(72) Inventors: NICHOLAS M. SAMPAS, Loveland, CO (US); Kristin Bernick, Loveland, CO (US)

(73) Assignee: AGILENT TECHNOLOGIES, INC., Loveland, CO (US)

(21) Appl. No.: 13/791,616

(22) Filed: Mar. 8, 2013

(51) Int. Cl. C12Q 1/68

(52) U.S. Cl. CPC C12Q 1/6876
USPC 435/6.11; 436/501; 536/24.3

(57) ABSTRACT

A composition comprising population of labeled oligonucleotides is provided herein. In some embodiments, the probes are of the formula: T₁-V-T₂, wherein: the nucleotide sequence of the V region varies in said population; the V regions of the different oligonucleotides hybridize to sites that are tiled across a sequence in a target nucleic acid; the T₁ and T₂ regions do not hybridize with said target nucleic acid; within each oligonucleotide of the population, the T₁ and T₂ regions are not complementary; and within each oligonucleotide of the population, the T₁ region is complementary to the T₂ region of at least one of other oligonucleotide of the population. Also provided is a method that comprises hybridizing the population of labeled oligonucleotides with a target nucleic acid to produce a complex.

### BACKGROUND

Oligonucleotide FISH (“oFISH”) has demonstrated utility in the field of cytogenetics for the discovery and validation of deletions, insertions, and rearrangements of genomic DNA. Detection of these types of events has applications in disease diagnostics for genomic disorders, prenatal testing and cancer. FISH probes have also been used to validate copy number variations in normal samples, and to differentiate the maternal and paternal origins of chromosomal alleles. FISH has also been used to detect messenger-RNA molecules (RNA-FISH) within individual cells enabling direct measurements of cellular heterogeneity. However, the inability of oligonucleotide FISH or conventional BAC-FISH to address smaller genomic aberrations or genomic variants sometimes limits these applications to larger genomic events. Genomic FISH probes are currently targeted towards genomic regions that are typically tens of kilobases, or more, whereas optical resolution, when it comes to metaphase cytogenetic imaging, can be as low as about five megabases. Consequently, it is sometimes difficult to resolve spatial relationships that are of sub-optical resolution, such as small inversions. On the other hand, multiple neighboring probes can be detected with reasonable sensitivity at a resolution beyond that of conventional optical microscopy. And, the more label molecules there are binding to a target region, the stronger the resulting signal. The sensitivity in cases of few target molecules or label molecules can be limited, especially for smaller target intervals. In certain circumstances, sensitivity can be limited in RNA FISH performed in both live and fixed cells due to the limited sizes of and accessibility of the target mRNA molecules due to both RNA structure and the complexes it forms with cellular proteins. RNA FISH performed in live cells has an added complexity in that the live cells cannot be washed. Thus any unbound probe can still fluoresce within the cell, which adds to the background signal.

### SUMMARY

This disclosure provides a composition comprising population of labeled oligonucleotides. In some embodiments, the probes are of the formula: T₁-V-T₂, where the nucleotide sequence of the V region varies in the population, the V regions of the different oligonucleotides hybridize to sites that are tiled across a sequence in a target nucleic acid, the T₁ and T₂ regions do not hybridize with the target nucleic acid, within each oligonucleotide of the population, the T₁ and T₂ regions are not complementary, and within each oligonucleotide of the population, the T₁ region is complementary to the T₂ region of at least one of other oligonucleotide of the population.

Also provided is a method comprising hybridizing the population of labeled oligonucleotides to a target nucleic acid to produce a complex comprising the target nucleic acid and a plurality of labeled oligonucleotides that are i. hybridized to sites tiled along the target nucleic acid and ii. hybridized to one another via their T₁ and T₂ regions, and detecting binding of the labeled population of oligonucleotides using the label of the oligonucleotides.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates an exemplary population of oligonucleotides.

FIG. 2 schematically illustrates the exemplary population of oligonucleotides shown in FIG. 1, hybridized to a target nucleic acid.

FIG. 3 schematically illustrates some general principles of the subject method.

FIG. 4 schematically illustrates a population of oligonucleotides that are labeled randomly or directly.

FIG. 5 schematically illustrates a population of oligonucleotides that are end-labeled.

FIG. 6 schematically illustrates a population of oligonucleotides that provide a FRET signal.

FIG. 7 illustrates a specific example of a complex that contains three oligonucleotides (two are only partially shown). The T₁ and T₂ regions of the oligonucleotides are fully complementary. (SEQ ID NOS: 1-4)

FIG. 8 illustrates a specific example of a complex that contains three oligonucleotides. The complementary part of one of the twist tie regions is only 11 basepairs in length, while the twist tie region in the other oligonucleotide is 18 basepairs in length, which creates a bulge of 7 bp between the duplex with the target and the duplex of the twist-tie. (SEQ ID NOS: 5, 2, 6, and 4)

FIG. 9 illustrates a specific example of a complex that contains three oligonucleotides. This embodiment is designed to produce a high resolution signal. In this embodiment, the separation of the activator dye molecules and reporter dye molecule is 9 bp. (SEQ ID NOS: 5, 2, 6, and 4)

FIG. 10 schematically illustrates a complex that uses molecular beacon twist-tie oligonucleotides. As shown, the beacon structure is built into the primer sequence at the 5′ end of each of the oligonucleotides.

FIG. 11 illustrates an embodiment of the method that uses short oligonucleotides as quenchers.

### DEFINITIONS

Before describing exemplary embodiments in greater detail, the following definitions are set forth to illustrate and define the meaning and scope of the terms used in the description.

Numeric ranges are inclusive of the numbers defining the range. Unless otherwise indicated, nucleic acids are written left to right in 5′ to 3′ orientation; amino acid sequences are written left to right in amino to carboxy orientation, respectively.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

The term “oligonucleotide” as used herein denotes a single-stranded multimer of nucleotide of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers. An oligonucleotide may be 10 to 20, 11 to 30, 31 to 40, 41 to 50, 51-60, 61 to 70, 71 to 80, 80 to 100, 100 to 150 or 150 to 200 nucleotides in length, for example.

The term “hybridization” or “hybridizes” refers to a process in which a nucleic acid strand anneals to and forms a stable duplex, either a homoduplex or a heteroduplex, under normal hybridization conditions with a second complementary nucleic acid strand, and does not form a stable duplex with unrelated nucleic acid molecules under the same normal hybridization conditions. The formation of a duplex is accomplished by annealing two complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions include hybridization at about 42 C in 50% formamide, 5×SSC, 5×Denhardt's solution, 0.5% SDS and 100 ug/ml denatured carrier DNA followed by washing two times in 2×SSC and 0.5% SDS at room temperature and two additional times in 0.1×SSC and 0.5% SDS at 42° C.

The term “in situ” refers to “inside a cell”. For example, the RNA being detected by in situ hybridization is present inside a cell. The cell may be permeabilized or fixed, for example.

The term “contacting” means to bring or put together. As such, a first item is contacted with a second item when the two items are brought or put together, e.g., by touching them to each other or combining them in the same solution.

The term “in situ hybridization conditions” as used herein refers to conditions that allow hybridization of a nucleic acid to a complementary nucleic acid, e.g., a sequence of nucleotides in a RNA or DNA molecule and a complementary oligonucleotide, in a cell. Suitable in situ hybridization conditions may include both hybridization conditions and optional wash conditions, which conditions include temperature, concentration of denaturing reagents, salts, incubation time, etc. Such conditions are known in the art.

The term “duplex,” or “duplexed,” as used herein, describes two complementary polynucleotides that are base-paired, i.e., hybridized together.

As used herein, the term “Tₘ” refers to the melting temperature of an oligonucleotide duplex at which half of the duplexes remain hybridized and half of the duplexes dissociate into single strands. The Tₘ of an oligonucleotide duplex may be experimentally determined or predicted using the following formula Tₘ=81.5+16.6(log₁₀[Na⁺])+0.41 (fraction G+C)−(60/N), where N is the chain length and [Na⁺] is less than 1 M. See Sambrook and Russell (2001; Molecular Cloning: A Laboratory Manual, 3^{rd} ed., Cold Spring Harbor Press, Cold Spring Harbor N.Y., ch. 10). Other formulas for predicting Tₘ of oligonucleotide duplexes exist and one formula may be more or less appropriate for a given condition or set of conditions.

The term “free in solution,” as used here, describes a molecule, such as a polynucleotide, that is not bound or tethered to another molecule.

The terms “plurality”, “population” and “collection” are used interchangeably to refer to something that contains at least 2 members. In certain cases, a plurality, population or collection may have at least 10, at least 100, at least 1,000, at least 10,000, at least 100,000, at least 10⁶, at least 10⁷, at least 10⁸ or at least 10⁹ or more members.

If two nucleic acids are “complementary”, they base pair with one with one another. The term “perfectly complementary” is used to describe a duplex in which each base of one of the nucleic acids base pairs with a complementary nucleotide in the other nucleic acid. In many cases, two sequences that are complementary have at least 10, e.g., at least 12 or 15 nucleotides of complementarity.

The term “digesting” is intended to indicate a process by which a nucleic acid is cleaved by a restriction enzyme or other enzyme, such as a nuclease. In order to digest a nucleic acid, a restriction enzyme and a nucleic acid containing a recognition site for the restriction enzyme are contacted under conditions suitable for the restriction enzyme to work. Conditions suitable for activity of commercially available restriction enzymes are known, and supplied with those enzymes upon purchase.

An “oligonucleotide binding site” refers to a site to which an oligonucleotide hybridizes in a target polynucleotide. If an oligonucleotide “provides” a binding site for a primer, then the primer may hybridize to that oligonucleotide or its complement.

In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure. The nucleotide sequences of the first strand of several exemplary mammalian chromosomal regions (e.g., BACs, assemblies, chromosomes, etc.) is known, and may be found in NCBI's Genbank database, for example.

The term “unique sequence”, as used herein, refers to nucleotide sequences that are different from one another, or their complements. For example, a first unique sequence has a different nucleotide sequence than a second unique sequence or its complement. Unless otherwise indicated, a unique sequence is only present in one polynucleotide in a sample.

The term “do not hybridize to each other”, as used herein in the context of nucleic acids that do not hybridize to each other, refers to sequences that been designed so that they do not anneal to one another under stringent conditions.

The term “similar to one another” in the context of a polynucleotide or polypeptide, means sequences that are at least 70% identical, at least 80% identical, at least 90% identical, or at least 95% identical, to one another.

The term “variable”, in the context of two or more nucleic acid sequences that are variable, refers to two or more nucleic acids that have different sequences of nucleotides relative to one another. In other words, if the polynucleotides of a population have a variable sequence, then the nucleotide sequence of the polynucleotide molecules of the population varies from molecule to molecule. The term “variable” is not to be read to require that every molecule in a population has a different sequence to the other molecules in a population.

The following description explains the formulas used in this disclosure. Certain polynucleotides described herein may be referred by a formula (e.g., “T₁-V-T₂”). Such formulas follow the established convention in that they describe a polynucleotide that is oriented in the 5′ to 3′ direction. The components of the formula, e.g., “T₁”, “V” and “T₂′” refer to separately definable sequences of nucleotides within a polynucleotide, where the sequences are linked together covalently such that a polynucleotide described by a formula is a single molecule. The components of the formula may be immediately adjacent to one another or spaced from one another in the single molecule. In certain cases, other sequence elements, may be provided by sequences that are between the components of a formula. Further, each of the various components of a formula may have functions in addition to those described herein. Following convention, the complement of a sequence shown in a formula will be indicated with a prime 0 such that the complement of sequence “V” will be “V”. Moreover, unless otherwise indicated or implicit from the context, a polynucleotide defined by a formula may have additional sequence at its 3′ end, its 5′ end or both the 3′ and 5′ ends.

As will be described in greater detail below, the T₁ region of one oligonucleotide may hybridize to the T₂ region of another oligonucleotide. The structure resulting from hybridization of a T₁ region of one oligonucleotide to the T₂ region of another oligonucleotide may be referred to as a “twist tie” in certain embodiments.

Other definitions of terms may appear throughout the specification.

### DESCRIPTION OF EXEMPLARY EMBODIMENTS

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

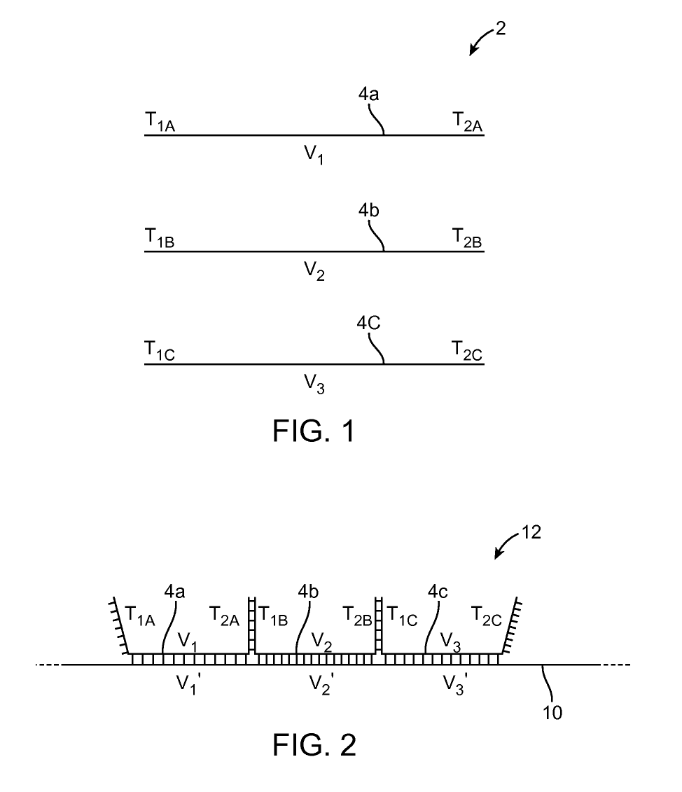

An exemplary population of labeled oligonucleotides of the formula T₁-V-T₂ is schematically illustrated in FIGS. 1 and 2. As would be apparent, the population of labeled oligonucleotides shown in these figures has three members. In practice, a subject population of oligonucleotides may contain many more, e.g., tens, hundreds or even thousands of members. With reference to FIGS. 1 and 2, population of labeled oligonucleotides 2 contains three oligonucleotides 4a, 4b and 4c. As shown, the nucleotide sequence of the V region varies in the population in that oligonucleotide 4a contains sequence V₁, oligonucleotide 4b contains sequence V₂ and oligonucleotide 4c contains sequence V₃. As shown in FIG. 2, hybridization of oligonucleotide 4a, 4b and 4c to target nucleic acid 10 produces complex 12. As shown in FIG. 2, the various V regions of the different oligonucleotides hybridize to sites that are tiled, i.e., next to one another, across a sequence in target nucleic acid 10. If the target is single stranded, then the various V regions should hybridize to that strand. If the target is double stranded (e.g., an intact chromosome), then there is no requirement that all of the oligonucleotides hybridize to the same strand. In some cases, all of the oligonucleotides of the population may hybridize to the same strand of the double stranded target. Alternatively, some of the oligonucleotides of the population may hybridize to one strand of the double stranded target and the remainder of the oligonucleotides of the population may hybridize to the other strand of the double stranded target. In this embodiment, the complementary T₁ and T₂ regions of oligonucleotides that are hybridized to the top strand should be different to the T₁ and T₂ regions of oligonucleotides that are hybridized to the top strand to prevent the formation of hybridization bridges across the two strands.

As shown in FIG. 2, the T₁ and T₂ regions do not hybridize with target nucleic acid 10 and, instead, hybridize with one another in the complex 12. As illustrated, the T₁ and T₂ regions within each oligonucleotide are not complementary to one another. For example, the T_{1A} region is not complementary to the T_{2A} region in oligonucleotide 4a, the T_{1B} region is not complementary to the T_{2B} region in oligonucleotide 4b, and the T_{1C} region is not complementary to the T_{2C} region in oligonucleotide 4c. However, within each oligonucleotide of the population, the T₁ region is complementary to the T₂ region of at least one of other oligonucleotide of the population such that the population of oligonucleotides 2 hybridize to target nucleic acid 10 in the manner shown in FIG. 2, i.e., to provide a complex in which the ends of the oligonucleotides that are bound to the target nucleic acid hybridize to the ends of other oligonucleotides that are bound to the target nucleic acid. As shown, in complex 12, the T_{2A} region of oligonucleotide 4a hybridizes to the T_{1B} region of oligonucleotide 4b and the T_{2B} region of oligonucleotide 4b hybridizes to the T_{1C} region of oligonucleotide 4c.

As will be described in greater below, the oligonucleotides of the population may be labeled by a single label at any position (e.g., at a 3′ or 5′ ends or at any position in between) or at multiple positions (at both the 3′ and 5′ ends, at least two internal sites, or at an end and at one or more internal sites). As will be described in greater detail below, if the oligonucleotides are labeled at multiple positions, the labels may be different from one another. In many embodiments, the label comprises an optically detectable moiety, e.g., a chromophore, fluorescent moiety, a quantum dot, etc.

As noted above, the oligonucleotides of a population are designed so that they hybridize to sites that are tiled across a sequence in the target nucleic acid. As such, there are two sub-populations of oligonucleotides in a subject population of oligonucleotides: a first sub-population of population of oligonucleotides and a second sub-population of population of oligonucleotides, where, in complex 12, oligonucleotides from the first sub-population and oligonucleotides from the second sub-population alternate with one another in “odd” and “even” positions, respectively. In certain embodiments, the first sub-population of oligonucleotides may have T₁ regions that are the same as one another and T₂ regions that are the same as one another. In these embodiments, the second sub-population of oligonucleotides may have T₁ regions that are the same as one another and T₂ regions that are the same as one another. Consistent with the above, the T₁ regions of the first sub-population hybridize to the T₂ regions of the second sub-population, and the T₂ regions of the first sub-population hybridize to the T₁ regions of the second sub-population.

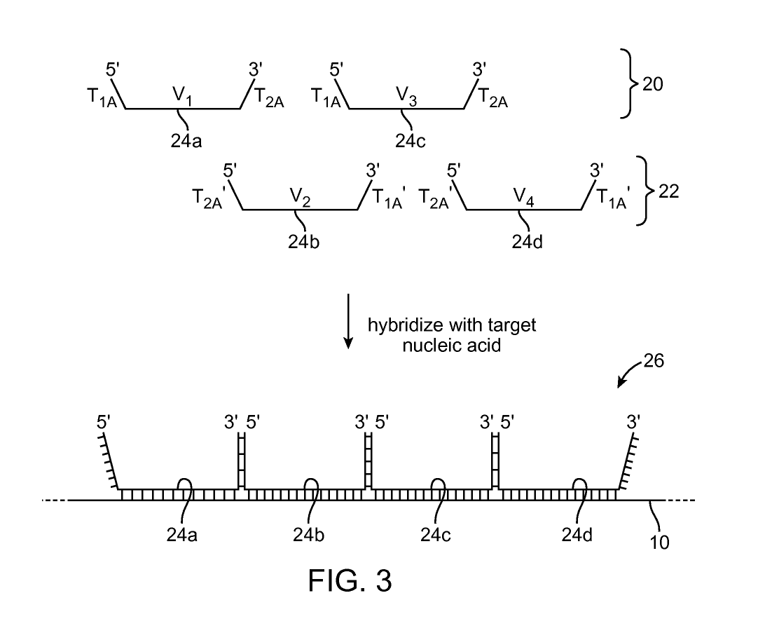

This embodiment is shown in FIG. 3. FIG. 3 shows a first sub-population of oligonucleotides 20 (which contains oligonucleotides 24a and 24c) and a sub-population of oligonucleotides 22 (which contains oligonucleotides 24b and 24d), where the oligonucleotides from the first sub-population and oligonucleotides from the second sub-population alternate with one another in “odd” and “even” positions in complex 26 that additionally contains target nucleic acid 10. As shown, oligonucleotides 24a and 24c have the same T₁ region (sequence T_{1A}) and the same T₂ region (sequence T_{2A}), whereas oligonucleotides 24b and 24d have the same T₁ region (sequence T_{2A′}, i.e., the complement of T_{2A}) and the same T2 region (sequence T_{1A}′, i.e., the complement of T_{1A}). When hybridized to target nucleic acid 10, complex 26 is produced. As described above, the oligonucleotides hybridize to sites that are tiled across a sequence in the target nucleic acid in an alternating way and the T₁ region of each of the oligonucleotides hybridize to the T₂ region in the adjacent oligonucleotide. In these embodiments, the population of oligonucleotides may comprise a first sub-population of oligonucleotides of formula T_{1A}-V-T_{2A} and a second sub-population of oligonucleotides of formula T_{1B}-V-T_{2B}, where the sequences of the T_{1A}, T_{2A}, T_{1B} and T_{2B} regions are different relative to one another and do not vary, the sequence of the T_{1A} region is complementary to the sequence of the T_{2B} region and the sequence of the T_{2A} region is complementary to the sequence of the T_{1B} region.

The nucleotide sequences of the T₁ and T₂ regions should be unique in the sense that they do not significantly hybridize to any other sequences in the sample. Further, when the T₁ and T₂ regions are used in a multiplex manner, they should be Tₘ-matched, where the term “Tₘ-matched” refers to a set of oligonucleotides that have Tₘs that are within a defined range, e.g., within 5° C. or 10° C. of one another. Sets of non-cross-hybridizing sequences are described in, e.g., US20070259357, US20030077607, US20100311957, and Brenner et al (Proc. Natl. Acad. Sci. 1992 89:5381-3). Further, computer algorithms for selecting non-crosshybridizing sets of sequences are described in Brenner (PCT Publications No. WO 96/12014 and WO 96/41011) and Shoemaker (Shoemaker et al., European Pub. No. EP 799897 A1 (1997)).

In many embodiments, the oligonucleotides in the population are designed so that the T₁ and complementary T₂ regions have a Tₘ that is at least 5° C., e.g., at least 10° C. or at least 15° C. lower than the Tₘ of V regions. In these embodiments, the T₁ and complementary T₂ regions of adjacent oligonucleotides may not hybridize to one another until the oligonucleotides have hybridized the target nucleic acid. This embodiment is illustrated in FIG. 3. In this embodiment, the complementary T₁ and T₂ regions in oligonucleotides 24a, 24b, 24c and 24d do not hybridize to one another until the V regions of those oligonucleotides hybridize to their respective targets. As would be apparent, the stringency of hybridization can be selected so that it disfavors hybridization of the complementary T₁ and T₂ regions unless those regions are brought into close proximity by hybridization of the V regions of the oligonucleotides to the target nucleic acid. Without wishing to be bound to any theory, it is believed that hybridization of the complementary T₁ and T₂ regions stabilizes the complex, thereby preventing the oligonucleotides from being separated from the target nucleic acid after they have hybridized. In certain cases the V regions may be, independently, from 15 to 200 bases in length, although, in practice any sequence that is greater than 20 nucleotides in length (e.g., 20 nt to 100 nt) may be used in certain circumstances. In some embodiments, the T₁ and T₂ sequences may be from 5 to 25 bases in length, e.g., 6 to 20 nucleotides or 7 to 15 nucleotides in length. In certain cases, the T₁ and complementary T₂ regions may have a Tₘ in the range of 40° C. to 60° C.

As will be exemplified below, the oligonucleotides may be labeled in a variety of different ways depending on how the binding of the oligonucleotides is going to be detected. In certain embodiments, each of the oligonucleotides may comprise at least two different fluorophores. In these embodiments, the identity of the fluorophores and their positioning in each of the oligonucleotides may be chosen so as to provide a for fluorescence resonance energy transfer (FRET) when two adjacent oligonucleotides are hybridized to the target nucleic acid. A wide variety of FRET pairs have been described previously (See, e.g., U.S. Pat. Nos. 6,008,373, 7,449,298, 5,925,517, 5,210,015, 5,487,972 and 5,763,181). Alternatively, the fluorophores can be chosen and positioned so as to provide a signal that has a resolution of less than 50 nm (e.g., 20 nm to 50 nm) when placed in proximity. In these embodiments, each probe may consist of a photo-switchable reporter fluorophore that can be cycled between fluorescent and dark states, and an activator that facilitates photo-activation of the reporter, as described in Bates et al (Science 2007 317: 1749-1753; incorporated by reference for disclosure of those methods). In these embodiments, each of the labeled oligonucleotides may contain a first label at the T₁ end of the oligonucleotide and a second label at the T₂ of the oligonucleotide, where the first and second labels produce a signal (e.g., a FRET signal or a high resolution signal, as discussed above) when placed in proximity. In this implementation, no FRET signal is produced until the adjacent oligonucleotides hybridize to the target nucleic acid.

Fluorescent moieties that can be linked to the oligonucleotides include, but are not limited to, xanthene dyes, e.g. fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′, 7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, ROX, Napthofluorescein, Texas Red, Napthofluorescein, Cy3, and Cy5, etc.

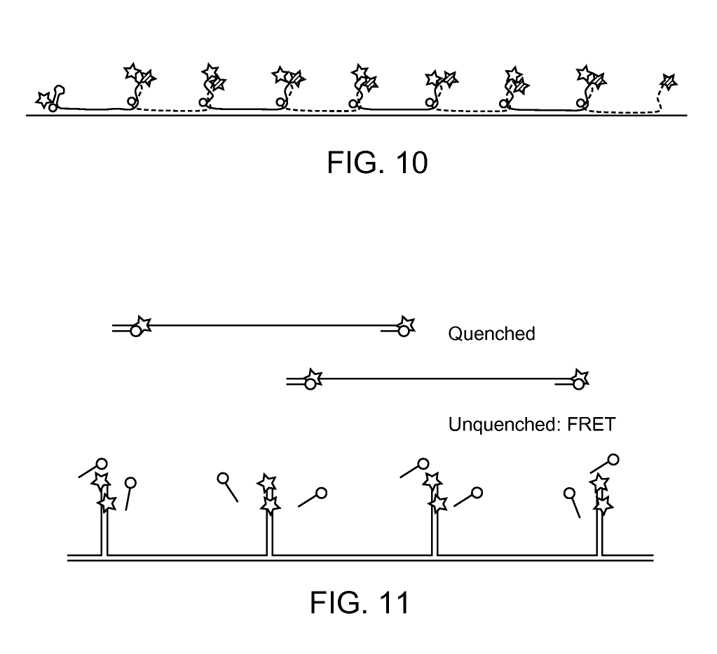

In certain embodiments and as will be shown in greater detail, in addition to a pair of FRET labels, the T₁ and/or T₂ regions of each of the labeled oligonucleotides (e.g., the end of the oligonucleotides that contain the donor fluorophore) may contain a hairpin when it is not hybridized to the T₁ or T₂ region of another of the labeled oligonucleotides. In these embodiments, the hairpin may additionally contain a quencher for one of the fluorophores (e.g., the donor fluorophore), where the quencher and the fluorophore are positioned so that fluorophore is quenched by the quencher until the T₁ and/or T₂ regions (whichever is attached to the fluorophore) is hybridized to the complementary T₁ and/or T₂ region in an adjacent oligonucleotide. This embodiment is illustrated in FIG. 10.

In certain embodiments, the different oligonucleotides of the population hybridize to sites that, collectively, define a contiguous sequence in the target nucleic acid (i.e., in other words, there are no gaps between the sites to which the V regions bind). In other embodiments, the different oligonucleotides of the population hybridize to sites that, collectively, are not defined by a contiguous sequence in the target nucleic acid. In these embodiments, there may be a gap of up to 10 nucleotides, e.g., up to 5 nucleotides or up to up to 2 nucleotides between the sites to which the oligonucleotides bind. The sequence to which the oligonucleotides of a population bind may be in the region of 100 bases to 50 kb in length, e.g., 1 kb to 20 kb or 200 bases to 5 kb) in length, and should contain no repeated sequences. In particular embodiments (and depending on the length of the sequence in the target nucleic acid) there may be at least 5, at least 10, at least 20, at least 50, at least 100, at least 500, at least 1,000 or at least 5,000 or more oligonucleotides in a population (e.g., 5 to 100 oligonucleotides). In certain embodiments, the oligonucleotides may comprise a non-hybridizing spacer sequence between the V region and the T₁ region and the V region and the T₂ region. This embodiment is illustrated in FIGS. 8 and 9.

As should be apparent, the composition in which the population of oligonucleotides described above (referred to as the “first” population of oligonucleotides) may comprise further populations (at least one other, at least 5 other, at least 10 other, at least 50 other, at least 100 other, or at least 1,000 or more other populations) of oligonucleotides that hybridize to sequences that are spaced from the sequences to which the first population of oligonucleotides binds. In these embodiments, the further populations of oligonucleotides may be labeled with the same label(s) as the first population of oligonucleotides described above. In other embodiments, the further populations of oligonucleotides may be labeled with the different label(s) relative to the first population of oligonucleotides described above. In some embodiments, hybridization of the first population of oligonucleotides and the second population of oligonucleotides may produce a multi-color signal.

A method for sample analysis is provided. In certain embodiments, this method may comprise hybridizing a population of labeled oligonucleotides as described above with a target nucleic acid to produce a complex comprising said target nucleic acid and a plurality of labeled oligonucleotides that are i. hybridized to sites tiled along said target nucleic acid and ii. hybridized to one another via their T₁ and T₂ regions; and detecting binding of said labeled population of oligonucleotides using the label of said oligonucleotides.

The target nucleic acid may be any type of nucleic acid, including genomic DNA, RNA (including unprocessed RNA and processed RNA) or cDNA. In certain cases, the hybridizing may be done in vitro on an isolated target nucleic acid. In other embodiments, the hybridizing may be done in situ and the target nucleic acid may be an intact chromosome or RNA. In some cases, the hybridizing may be done in situ and the target nucleic acid is in a living cell (see, e.g., Wiegang et al Methods Mol. Biol. 2010 659:239-46; Dirks et al Methods 2003 29: 51-7; Lorenz RNA 2009 15:97-103 and U.S. Pat. Nos. 6,586,240 and 5,728,527). In embodiments that use living cells, the oligonucleotides may be introduced into the cells by microinjection, using cationic transfection agents, electroporation, by permeating the cell membrane by, e.g., using streptolysin O (SLO), or by conjugating the oligonucleotides to cell-penetrating peptides (CPPs), for example.

Certain hybridization methods used herein include the steps of fixing a biological or non-biological sample (e.g., intact chromosomes or cells), hybridizing oligonucleotides to RNA or DNA molecules (e.g., RNAs or chromosomes) contained within the fixed sample, and washing the hybridized sample to remove non-specific binding. In situ hybridization assays and methods for sample preparation are well known to those of skill in the art and need not be described in detail here. Such methods can be found in, for example, Amann R. et al., 1995, Microbiol. Rev. 59(1): 143-69; Bruns and Berthe-Corti, 1998, Microbiology 144, 2783-2790; Vesey G. et al., 1998, J. App. Microbiol. 85, 429-440; and Wallner G. et al., 1995, Appl. Environ. Microbiol. 61(5): 1859-1866, and US20100081131, which are incorporated by reference herein.

The sample is then contacted with labeled polynucleotides under in situ hybridizing conditions, where “in situ hybridizing conditions” are conditions that facilitate annealing between a nucleic acid and the complementary nucleic acid. Hybridization conditions vary, depending on the concentrations, base compositions, complexities, and lengths of the probes, as well as salt concentrations, temperatures, and length of incubation. For example, some in situ hybridizations may be are performed in hybridization buffer containing 1-2×SSC, 50% formamide, and blocking DNA to suppress non-specific hybridization. In general, hybridization conditions include temperatures of about 25° C. to about 55° C., and incubation times of about 0.5 hours to about 96 hours. Suitable hybridization conditions for a library of oligonucleotides and target microbe can be determined via experimentation which is routine for one of skill in the art.

In certain embodiments, cells can be harvested from a biological or non-biological sample using standard techniques. For example, cells can be harvested by centrifuging a sample and resuspending the pelleted cells in, for example, phosphate-buffered saline (PBS). After re-centrifuging the cell suspension to obtain a cell pellet, the cells can be fixed in a solution such as an acid alcohol solution, an acid acetone solution, or an aldehyde such as formaldehyde, paraformaldehyde, or glutaraldehyde. For example, a fixative containing methanol and glacial acetic acid in a 3:1 ratio, respectively, can be used as a fixative. A neutral buffered formalin solution also can be used (e.g., a solution containing approximately 1% to 10% of 37-40% formaldehyde in an aqueous solution of sodium phosphate). Slides containing the cells can be prepared by removing a majority of the fixative, leaving the concentrated cells suspended in only a portion of the solution. Methods for fixing cells are known in the art and can be adapted to suit different types of microbes, if needed. Determination of suitable fixation/permeabilization protocols are carried out routinely in the art.

A hybridized sample can be read using a variety of different techniques, e.g., by microscopy, such as light microscopy, fluorescent microscopy or confocal microscopy. In embodiments in which oligonucleotides are labeled with a fluorescent moiety, reading of the contacted sample to detect hybridization of labeled oligonucleotides may be carried out by fluorescence microscopy. Fluorescent microscopy, including confocal microscopy and structured illumination microscopy (SIM), has an added advantage of distinguishing multiple labels even when the labels overlap spatially. Methods of reading fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361. In certain embodiments, the microscope may be a high resolution microscope such as that described in Bates et al (Science 2007 317: 1749-1753).

In certain embodiments, the signal obtained from performing the method may be compared with that of a reference sample, e.g., a cell chromosome a healthy or wild-type organism. Briefly, the method comprises contacting under in situ hybridization conditions a test sample with a plurality of probes described above and contacting under in situ hybridization conditions a reference chromosome with the same plurality probes. After hybridization, the emission spectra created from the unique binding patterns from the test sample are compared against those of the reference sample.

In one embodiment, a binding pattern obtained from a test sample may be compared to the pattern of binding of the same probes with a reference sample. The binding pattern of the reference sample may be determined before, after or at the same time as the binding pattern for the test sample. This determination may be carried out either manually or in an automated system. In certain cases, the signal associated with the test sample can be compared to the binding pattern that would be expected for known deletions, insertions, translocation, fragile sites and other more complex rearrangements, and/or refined breakpoints. The matching may be performed by using computer-based analysis software known in the art. Determination of identity may be done manually (e.g., by viewing the data and comparing the signatures by hand), automatically (e.g., by employing data analysis software configured specifically to match optically detectable signature), or a combination thereof.

In certain embodiments, oligonucleotide probes may be designed using methods set forth in US20040101846, U.S. Pat. No. 6,251,588, US20060115822, US20070100563, US20080027655, US20050282174, patent application Ser. No. 11/729,505, filed March 2007 and patent application Ser. No. 11/888,059, filed Jul. 30, 2007 and references cited therein, for example. In certain embodiments, the oligonucleotides may be synthesized in an array using in situ synthesis methods in which nucleotide monomers are sequentially added to a growing nucleotide chain that is attached to a solid support in the form of an array. Such in situ fabrication methods include those described in U.S. Pat. Nos. 5,449,754 and 6,180,351 as well as published PCT application no. WO 98/41531, the references cited therein, and in a variety of other publications. In one embodiment, the oligonucleotide composition may be made by fabricating an array of the oligonucleotides using in situ synthesis methods, and cleaving oligonucleotides from the array. The oligonucleotides may be amplified prior to use (e.g., by using PCR using primer sites that are at the terminal regions of the oligonucleotides, or by using polymerase promoter, e.g., a T7 polymerase promoter, that is at a terminal region of the oligonucleotides).

Some embodiments of the method increase the sensitivity of the detection of fluorescent probes by making their hybridization more stable, increasing the numbers of end-labeled oligonucleotides within a finite span and by using FRET to increase the signal with respect to the background when the probe oligonucleotides are bound to the same target molecule. Because this method is compatible with some high-resolution microscopy techniques (i.e. Stochastic Optical Reconstruction Microscopy or STORM; see Bates et al Science 2007 317: 1749-1753), this approach can enable detection of events that may be beyond the resolution of conventional optical microscopy. This approach can also increase the sensitivity beyond that of conventional fluorescence microscopy. Several distinct embodiments are described below.

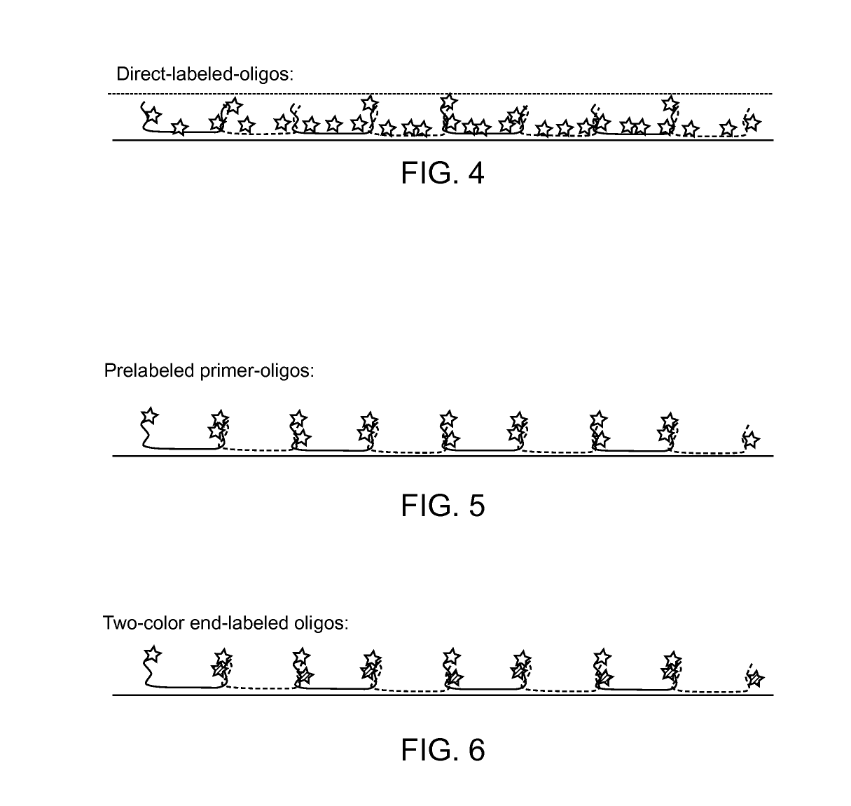

In one embodiment, the method can be practiced by designing cross-complementary oligonucleotides in such a way that each end of each oligonucleotide hybridizes with the nearby end of its adjacent oligonucleotides. The main portion of each oligonucleotide is designed so as to hybridize to the target sequence. Additionally, each end has additional sequence that does not exist in a host organism, but is complementary to the end of the adjacent oligonucleotides, with consecutive complementary sequences of between 5 and 40 base pairs, e.g., 6 to 20 bp. The optimal length of the complementary regions (which in certain cases can be referred to as “twist ties”) can be optimized to promote the formation and stability of complex of target and oligonucleotides, but should be sufficiently short so that the oligonucleotides do not bind each other in solution in a way that inhibits their binding to the target sequence. The oligonucleotides may be randomly labeled, e.g. using an enzyme, or direct labeled, e.g. ULS labeling, as depicted in FIG. 4. As shown in FIG. 4, the labels can be of a single type. In practice, the various populations can each be labeled independently with different dye molecules.

In some embodiments, each oligonucleotide can be labeled at or near one end or both ends, as shown in FIG. 5. In this embodiment, the 5′ end of the oligonucleotides can be labeled by means of a prelabeled primer and by the use of an enzyme to extend the primer by means of a template molecule. This can be done bidirectionally, e.g. by using PCR, or unidirectionally using a polymerase to make single directional copies of a DNA template. The 3′ end can be labeled directly using standard enzymatic methods, such as terminal deoxynucleotidyl transferase or T4 RNA ligase. If amplification methods are used in the labeling process and single strands are ultimately required for experiments, primers for the undesired strand can be phosphorylated allowing the resulting amplified phosphorylated strands to be degraded by lambda exonuclease, resulting in the desired strand as single stranded DNA. Oligonucleotides that are labeled in a single label using these methods are depicted in FIG. 5.

In certain cases the alternating arranged oligonucleotides can be labeled at both ends in such a way that they provide adjacent donor and acceptor molecules. This enables sensitive detection, stable multiplex formation and shorter oligonucleotides probes for high density labeling. This configuration (illustrated in FIG. 6) should make possible the detection of short genomic intervals and RNA messages within the cytoplasm of both fixed and live cells. In this embodiment, one end, e.g. 5′-end, of a first probe oligonucleotide within a sub-population of oligonucleotide may be labeled with a donor fluorophore and the other end, e.g. the 3′-end, of a second oligonucleotides is labeled with an acceptor fluorophore. The twist tie regions of these oligonucleotides are adjacent, or nearly adjacent, on the target molecule. A few base-pairs can be left in between the labels to minimize contact quenching which can result from direct contact between the donor and acceptor, resulting in reduced signal. The close proximity of the two dye molecules allows them to transfer energy by FRET (Fluorescence Resonance Energy Transfer), which occurs when a donor dye moiety is proximal to the acceptor moiety, e.g., when it is located within approximately 5 nm of an acceptor molecule. Efficient detection is possible when the donor fluorophore is excited near the peak of its absorbance band, the emission of the donor is within the absorbance band of the acceptor and light is detected over the emission band acceptor fluorophore. In this configuration, each oligonucleotide may have on average no more than a single FRET pair. A somewhat lower ratio will result where small genomic regions are omitted, for example to avoid repetitive elements or other uninformative sequences.

In certain cases one could use two or more pools of oligonucleotides, so that distinct primers can be used to create common sequences across each pool of oligonucleotides while eliminating the use of complementary primer sequence or subsequences to both amplify the source DNA and form the complementary sequences. As an alternative, each adjacent oligonucleotide pair can have its own specific twist tie sequence. However, the use of many distinct sequences may negate some of the efficiency advantages of the commonly primed amplification of complex libraries of probe sequences. Each sub-population of oligonucleotides can be made using a single pair of primers, and two or more sub-populations can be used to make alternating (odd and even) sets of oligonucleotides (as described above). The use of distinct twist-ties for each sub-population eliminates the hybridization of complementary primer sequences or subsequences needed to construct the sequences.

The FRET-based embodiments of this method may be practiced using two different configurations of dye molecules, both involving using two or more distinct sub-populations of oligonucleotides. Consider a population of oligonucleotides designed over a contiguous span of a target strand. If the oligonucleotides are numbered in order along the target, then one can refer to two sets of alternating probes as the odd sub-population (with oligonucleotides 1, 3, 5, . . . ) and an even sub-population, (with oligonucleotides 2, 4, 6, . . . ). In a first embodiment, the odd sub-populations is labeled on (or near) both ends with a first dye molecule, and the even sub-populations is also labeled on (or near) both ends with a second dye molecule. One of these dye molecules is a donor and the other is an acceptor and when the two sets hybridize substantially to the same target molecule then they can be detected with high sensitivity by enhanced emission of light over the emission band of the acceptor molecule when excited at the donor absorbance wavelength. In a second embodiment, the odd and even sub-populations are labeled similarly, but with a first dye at the 5′ end and a second dye at the 3′ end of all molecules irrespective of whether they are in even or odd sub-populations. Indeed, they may be derived from the same sub-population, depending on the labeling method.

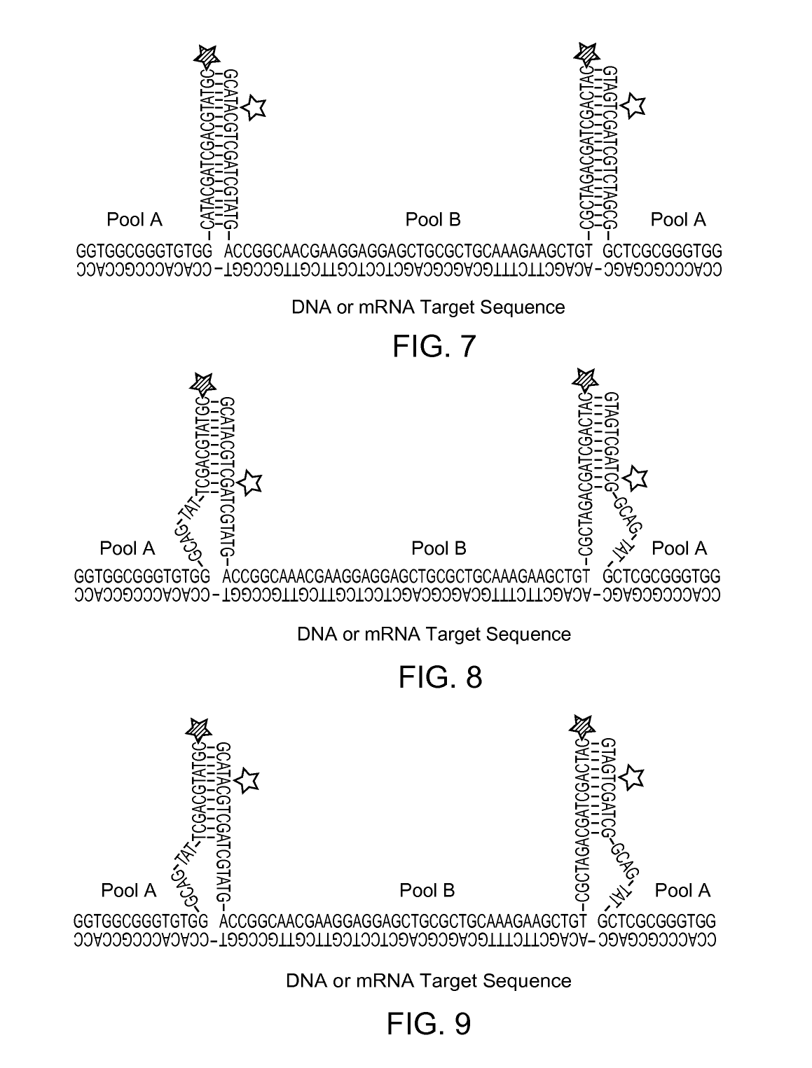

Some FRET embodiments are illustrated in FIGS. 7 and 8. FIG. 7 illustrates an example of twist tie sequences that are complementary over the full 18-bp length. This arrangement may be advantageous for complex stability. An alternative embodiment employs the use of a multibase mismatch within the complementary structures on both ends of the oligonucleotides. One such example is depicted in FIG. 8. In this example, the mismatches are depicted as bulges adjacent to the junction while the matched portions of the twist ties are at the ends of the twist-tie sequences. This geometry could be reversed so that the oligonucleotides are complementary at the junction but with frayed (unpaired) oligonucleotide ends. This configuration provides for the 3′-labeling of the oligonucleotides and the nearby internal label near the 5′ end of the complementary sequence. This combination is straightforward to construct using well established labeling methods. Primer sequences of different lengths and internal base positions can be used in both cases to move the positions of each of the dye molecules, providing many degrees or freedom for optimization. The geometry for high-resolution or single-molecule detection is nearly identical to that of the FRET embodiment described above. In certain cases, an offset of about 9 bases can used between an activator dye (e.g., Cy3), and the reporter dye (Cy5). This is shown in FIG. 9.

Another alternative embodiment combines the FRET-related embodiments described above with molecular beacon probes. This approach uses primer sequences that form moderately stable hairpin structures at one or both ends of the oligonucleotides that bring a quencher into close proximity with the dye molecule in order to quench its fluorescence when not in a twist-tie conformation, such as when it is not bound to a target molecule. However, when adjacent probes are bound to the target sequence, these end sequence form the twist-tie structure described above, unfolding the hairpin and bringing the two labels in close proximity. These twist-tie sequences may be designed to form stable hairpins under the hybridization conditions used, such as used for live cells, and the hairpins become destabilized at somewhat higher temperatures during the annealing phase of the in situ hybridization reaction. The hairpin can be at either or both ends of the oligonucleotides. However, it is more practical to construct these structures at the 5′ end where they can be synthesized into the primer sequence before they are amplified. This configuration is depicted in FIG. 10. The hairpin structures can be used to quench the donor or the acceptor or both. The choice of which configuration to use depends on the signal to noise of the assay as well as the ease and practicality of manufacturing oligonucleotides with these quenched constructs.

Finally, in the embodiment shown in FIG. 11, the probes may quenched by short oligonucleotides containing quencher molecules and are in the “off” state. In the presence of target, the barcodes base pair with one another and the quencher oligonucleotides are displaced, thereby turning the probes “on”. In the embodiment shown, short oligonucleotides containing quenchers at one end are designed to hybridize to the barcode regions of the oligonucleotide probes in such a way that the quencher is in close proximity (<5 nm) to the fluorophore. This results in quenching the fluorophores in the absence of target. When the probes bind their target (RNA or DNA), the barcodes form a more stable structure that displaces the shorter oligonucleotides resulting in the fluorophores being turned “on”. In certain cases, the quencher oligonucleotide can be mixed with the probe oligonucleotides, allowed to hybridize, and then hybridized to its target in quenched form.

In some embodiments, the oligonucleotides may be RNAs, linked nucleic Acids (LNAs), xenonucleic acids (XNAs) or peptide nucleic acids (PNAs). Each of these chemical moieties adds to the thermodynamic stability of the duplexes. Currently, there are six distinct classes of XNAs, each of which involve the use of alternative sugar molecules in place of deoxyribose in the phosphate backbone of DNA. XNAs are particularly suitable for application to live cell FISH because they are not degraded by the same enzymatic and chemical processes that degrade DNA and RNA in live cells. These XNAs can specifically base-pair or hybridize to conventional DNA and RNA. Further, some of them are good substrates for enzymes, such as polymerases and transcriptases that are used in amplification and extension of DNA. Additionally, some modified enzymes can preferentially amplify and extend some XNAs over the natural forms. XNAs may be used because of its base-pairing equivalence with natural DNA. Alternatively, left-handed DNA can used in the twist tie structures at the ends as they will not non-specifically hybridize with any natural DNA or RNA within the cell or any other non-targeted nucleic acids, but it will form equivalently stable duplexes with itself. These left-handed structures (of any nucleic backbone) need to be chemically bound or ligated to conventional DNA or XNAs for use as a probe.

Oligonucleotides that contain modified nucleotides, such as 2-prime-methoxinucleic acids (2′OMe-DNA) and a-L-threofuranosyl nucleic acid (TNA) may also be employed. 2′OMe-DNA nucleic acids are good template for some natural reverse transcriptases. Further, TNA has been demonstrated as a viable substrate for some polymerases, while only showing only limited reverse transcription. Modified nucleic acids using 2′OMe-DNA have been demonstrated as molecular beacons in a dual-FRET rRNA assay.

### Kits

Also provided by this disclosure is a kit for practicing the subject method, as described above. The various components of the kit may be present in separate containers or certain compatible components may be pre-combined into a single container, as desired. In some embodiments, the kit may comprise a composition comprising population of labeled oligonucleotides of the formula: T₁-V-T₂, wherein: i. the nucleotide sequence of the V region varies in said population; ii. the V regions of the different oligonucleotides hybridize to sites that are tiled across a sequence in a target nucleic acid; iii. the T₁ and T₂ regions do not hybridize with said target nucleic acid; iv. within each oligonucleotide of the population, the T₁ and T₂ regions are not complementary; and iv. within each oligonucleotide of the population, the T₁ region is complementary to the T₂ region of at least one other oligonucleotide of the population. In addition, the kit may contain reagents for hybridizing the oligonucleotides to a sample.

In addition to above-mentioned components, the subject kits may further include instructions for using the components of the kit to practice the subject methods, i.e., to provide instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

1. A composition comprising population of labeled oligonucleotides of the formula:
T₁-V-T₂,
wherein:
i. the nucleotide sequence of the V region varies in said population;
ii. the V regions of the different oligonucleotides hybridize to sites that are tiled across a sequence in a target nucleic acid;
iii. the T₁ and T₂ regions do not hybridize with said target nucleic acid;
iv. within each oligonucleotide of the population, the T₁ and T₂ regions are not complementary; and
iv. within each oligonucleotide of the population, the T₁ region is complementary to the T₂ region of at least one of other oligonucleotide of the population.

2. The composition of claim 1, wherein each of said labeled oligonucleotides comprises a single type of label.

3. The composition of claim 1, wherein each of said labeled oligonucleotides comprises at least two different types of label.

4. The composition of claim 1, wherein each of said labeled oligonucleotides comprises a fluorescent label.

5. The composition of claim 1, wherein each of said labeled oligonucleotides is labeled at both the T₁ and the T₂ regions.

6. The composition of claim 5, wherein the different labels produce a FRET signal when they are proximal to one another.

7. The composition of claim 1, wherein the different labels produce a signal that has a resolution of less than 50 nm when they are proximal to one another.

8. The composition of claim 1, wherein the T₁ and/or T₂ regions of each of said labeled oligonucleotides comprises a hairpin when it is not hybridized to the T₁ or T₂ region of another of said labeled oligonucleotides.

9. The composition of claim 8, wherein said hairpin contains a fluorophore and a quencher for said fluorophore, and wherein said quencher quenches said fluorophore in said hairpin.

10. The composition of claim 1, wherein the different oligonucleotides of the population hybridize to sites that, collectively, span a contiguous sequence in said target nucleic acid.

11. The composition of claim 1, wherein said oligonucleotides comprise non-hybridizing spacer sequences between the V region and the T₁ region and the V region and the T₂ region.

12. The composition of claim 1, wherein the population of labeled oligonucleotides comprises:
a first sub-population of oligonucleotides of formula T_{1A}-V-T_{2A}; and
a second sub-population of oligonucleotides of formula T_{1B}-V-T_{2B},
wherein the sequences of the T_{1A}, T_{2A}, T_{1B} and T_{2B} regions are different relative to one another and do not vary, the sequence of the T_{1A} region is complementary to the sequence of the T_{2B} region and the sequence of the T_{2A} region is complementary to the sequence of the T_{1B} region.

13. The composition of claim 1, wherein said T₁ and T₂ regions are, independently, 5 to 25 in length.

14. The composition of claim 1, wherein said population comprises at least 10 members.

15. A method comprising:
hybridizing a population of labeled oligonucleotides of claim 1 with a target nucleic acid to produce a complex comprising said target nucleic acid and a plurality of labeled oligonucleotides that are i. hybridized to sites tiled along said target nucleic acid and ii. hybridized to one another via their T₁ and T₂ regions; and
detecting binding of said labeled population of oligonucleotides using the label of said oligonucleotides.

16. The method of claim 15, wherein said target nucleic acid is genomic DNA or RNA.

17. The method of claim 15, wherein said hybridizing is done in vitro on an isolated target nucleic acid.

18. The method of claim 15, wherein said hybridizing is done in situ and said target nucleic acid is an intact chromosome.

19. The method of claim 18, wherein said hybridizing is done in vivo and said target nucleic acid is in a living cell.

20. A kit comprising a population of labeled oligonucleotides of claim 1.
